(19) United States
(12) Patent Application Publication
(10) Pub. No.: US 2012/0202888 A1
(43) Pub. Date: Aug. 9, 2012

(54) BIOMARKERS OF PROSTATE CANCER AND PREDICTING MORTALITY

(75) Inventor: Jing MA, Belmont, MA (US)

(73) Assignee: THE BRIGHAM AND WOMEN'S HOSPITAL, INC., Boston, MA (US)

(21) Appl. No.: 13/271,164

(22) Filed: Oct. 11, 2011

Related U.S. Application Data
(60) Provisional application No. 61/391,356, filed on Oct. 8, 2010.

(51) Int. Cl. A61K 31/155; A61P 3/10; G01N 33/566

(52) U.S. Cl. 514/635; 435/7.92

(57) ABSTRACT

Measurement of circulating C-peptide levels is useful for the prognostic evaluation of subjects, in particular for the prediction of adverse clinical outcomes, e.g., mortality, and the detection of fatal prostate cancer.

### CROSS REFERENCE TO RELATED APPLICATION

This application claims the benefit of U.S. Provisional Application Ser. No. 61/391,356, filed on Oct. 8, 2010, which is incorporated herein by reference in its entirety.

### FEDERALLY SPONSORED RESEARCH OR DEVELOPMENT

This invention was made with Government support under Grant Number R01-CA42182 awarded by National Institutes of Health. The Government has certain rights in the invention.

### TECHNICAL FIELD

The invention relates to methods evaluating risk of death from prostate cancer and methods of predicting risk of developing fatal prostate cancer by measuring circulating levels of C-peptide in combination with other biomarkers.

### BACKGROUND

A major challenge in prostate cancer (PC) research is distinguishing aggressive from indolent disease. Although the D'Amico risk stratification is helpful and widely used to guide PC treatment (D'Amico et al., JAMA 280:969-74, 1998), it relies on a few standard clinical parameters (prostate specific antigen (PSA), stage, and grade) and cannot always reliably distinguish patients who will die from PC from those who do not. With such uncertainty for future outcomes, most D'Amico low-risk patients choose radical treatment, leading to over-treatment and unnecessary side effects, preventing PC-specific mortality only in a small minority. Similarly, among patients presently classified as intermediate-high risk, some cancers are unlikely to progress while others may be destined to recur despite aggressive multi-modality therapy. There is an urgent need for additional biologically relevant markers to improve prognostication beyond algorithms based solely on PSA, stage, and grade. Ideally, such biomarkers could also provide clinical guidance for alternative or novel treatments.

### SUMMARY

The present invention is based, at least in part, on the discovery that adding risk scores based on levels of C-peptide, body-mass-index (BMI) ratio, or both C-peptide and BMI into an “energetic risk” score (BMI≧25 kg/m² and high C-peptide; or BMI≧30 kg/m²) to D'Amico risk groups significantly improved prediction of PC-specific and all-cause mortality, and identifies certain D'Amico “low-risk” patients who may be poor candidates for active surveillance.

In one aspect, the invention features methods for evaluating risk of mortality for a subject with prostate cancer. The methods include:

(i) providing a sample from the subject;

(ii) determining one or both of a level of C-peptide in the sample to obtain a test C-peptide value or determining the subject's body-mass-index ratio (BMI); and

(iii) determining the subject's D'Amico risk, i.e., by determining the subject's level of prostate-specific antigen (PSA), Gleason score, and clinical stage, wherein:
(a) a subject with a level of PSA less than 10 μg/mL, a Gleason score equal to or less than 6, and a clinical T1 or T2 stage has a low D'Amico risk;
(b) a subject with a level of 10-20 μg PSA/mL, a Gleason score of 7, or a clinical T1 or T2 stage has an intermediate D'Amico risk; and
(c) a subject with a level of greater than 20 μg PSA/mL, a Gleason score equal to or greater than 8, or a clinical T3 stage has a high D'Amico risk;
   wherein a subject with a test C-peptide value above a reference level of C peptide, a BMI equal to or greater than 25 kg/m², and an intermediate or high D'Amico risk has an elevated risk of mortality; a subject with.

In some embodiments, a subject with a test C-peptide value is in the highest quartile compared to a level of C-peptide in a reference group, a BMI of equal or greater than 25 kg/m², and an intermediate or high D'Amico risk has an elevated risk of mortality.

In some embodiments, a subject with a BMI of equal or greater than 30 kg/m² and an intermediate or high D'Amico risk has an elevated risk of mortality.

In some embodiments, the risk of mortality is from prostate cancer.

In some embodiments, the risk of mortality is within a specified time period, e.g., 1 year, 2 years, 5 years, or 10 years.

In another aspect, the invention provides methods for predicting risk of developing fatal prostate cancer in a subject. The methods include:

(i) providing a sample from the subject;

(ii) determining one or both of a level of C-peptide in the sample to obtain a test C-peptide value or determining the subject's body-mass-index ratio (BMI); and

(iii) determining the subject's D'Amico risk by determining the subject's level of prostate-specific antigen (PSA), Gleason score, and clinical stage, wherein:

(a) a subject with a level of PSA less than 10 μg/mL, a Gleason score equal to or less than 6, and a clinical T1 or T2 stage has a low D'Amico risk;

(b) a subject with a level of 10-20 μg PSA/mL, a Gleason score of 7, or a clinical T1 or T2 stage has an intermediate D'Amico risk; and

(c) a subject with a level of greater than 20 μg PSA/mL, a Gleason score equal to or greater than 8, or a clinical T3 stage has a high D'Amico risk;

wherein a subject with a test C-peptide value above a reference level of C peptide, a BMI equal to or greater than 25 kg/m², and an intermediate or high D'Amico risk has an elevated risk of developing fatal prostate cancer.

In some embodiments, a subject with a test C-peptide value is in the highest quartile compared to a level of C-peptide in a reference group, a BMI of equal or greater than 25 kg/m², and an intermediate or high D'Amico risk has an elevated risk of developing fatal prostate cancer.

In some embodiments, a subject with a BMI of equal or greater than 30 kg/m² and an intermediate or high D'Amico risk has an elevated risk of developing fatal prostate cancer.

In a further aspect, the invention provides methods for selecting an appropriate therapy for a subject with prostate cancer. The methods include:

(i) providing a sample from the subject;

(ii) determining one or both of a level of C-peptide in the sample to obtain a test C-peptide value or determining the subject's body-mass-index ratio (BMI); and

(iii) determining the subject's D'Amico risk by determining the subject's level of prostate-specific antigen (PSA), Gleason score, and clinical stage, wherein:

(a) a subject with a level of PSA less than 10 μg/mL, a Gleason score equal to or less than 6, and a clinical T1 or T2 stage has a low D'Amico risk;

(b) a subject with a level of 10-20 μg PSA/mL, a Gleason score of 7, or a clinical T1 or T2 stage has an intermediate D'Amico risk; and

(c) a subject with a level of greater than 20 μg PSA/mL, a Gleason score equal to or greater than 8, or a clinical T3 stage has a high D'Amico risk;

wherein a subject with a test C-peptide value above a reference level of C peptide (e.g., in the highest quartile compared to a level of C-peptide in a reference group), a BMI equal to or greater than 25 kg/m² (e.g., above 30 kg/mg²), and an intermediate or high D'Amico risk has an elevated risk of developing fatal prostate cancer, and is treated aggressively, e.g., with one or more of surgery (e.g., castration), radiation therapy, hormone therapy, chemotherapy, and biologic therapy.

In a further aspect, the invention provides methods for selecting an appropriate therapy for a subject with prostate cancer. The methods include:

providing a sample from the subject;

determining a level of C-peptide in the sample; and

selecting a therapy comprising an anti-diabetic or insulin-lowering drug (e.g., selected from the group consisting of metformin, phenformin, buformin, and proguanil) for a subject who has a level of C-peptide above, or at or above, a reference level, or selecting a therapy lacking an anti-diabetic or insulin-lowering drug for a subject who has a level of C-peptide below a reference level.

In some embodiments, the methods for selecting a therapy as described herein further include administering the selected therapy to the subject.

In yet another aspect, the invention features methods of monitoring the efficacy of a therapeutic intervention for prostate cancer. The methods include:

(i) providing an initial sample from the subject;

(ii) determining one or both of a level of C-peptide in the sample to obtain a test C-peptide value or determining the subject's body-mass-index ratio (BMI); and

(iii) determining the subject's D'Amico risk by determining the subject's level of prostate-specific antigen (PSA), Gleason score, and clinical stage, wherein:
(a) a subject with a level of PSA less than 10 μg/mL, a Gleason score equal to or less than 6, and a clinical T1 or T2 stage has a low D'Amico risk;
(b) a subject with a level of 10-20 μg PSA/mL, a Gleason score of 7, or a clinical T1 or T2 stage has an intermediate D'Amico risk; and
(c) a subject with a level of greater than 20 μg PSA/mL, a Gleason score equal to or greater than 8, or a clinical T3 stage has a high D'Amico risk;

(iv) administering a treatment to the subject, e.g., a treatment for prostate cancer, and/or a treatment comprising administration of a therapy comprising an anti-diabetic or insulin-lowering drug (e.g., selected from the group consisting of metformin, phenformin, buformin, and proguanil);

(v) determining a level of C-peptide, and optionally a level of PSA, in a subsequent sample obtained after administration of the treatment;

(vi) comparing the level of C-peptide, and optionally PSA, in the initial and subsequent samples;

wherein a decrease in a level of C-peptide and/or PSA in the sample indicates that the treatment is effective in treating PC in the subject.

In some embodiments of the methods described herein, the sample comprises serum, plasma, whole blood, or urine.

A “subject” as described herein can be any subject, e.g., a subject having prostate cancer. For example, the subject can be any mammal, such as a human, including a human cancer patient. Exemplary nonhuman mammals include a nonhuman primate (such as a monkey or ape), a mouse, rat, goat, cow, bull, pig, horse, sheep, cat, and dog. In some embodiments, the subject is a human.

An “anti-diabetic or insulin-lowering drug” as used herein is an agent that reduces insulin in a subject. Anti-diabetic or insulin-lowering drugs are known in the art and include metformin, troglitazone, rosiglitazone, phenformin, buformin, and proguanil.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F are a series of six graphs showing adjusted hazard ratio (HR) and cumulative incidence of death from prostate cancer (A, B, and C) and from any cause (D, E, and F), *P<0.05.

FIG. 2 is a pair of graphs showing the difference in post-prandial serum C-peptide levels before- and after-ADT with (right panel) or without (left panel) metformin.

### DETAILED DESCRIPTION

As described herein, a combination of energetic risk and D'Amico risk is useful in the prognostic evaluation of subjects with PC. Compared to D'Amico risk alone, incorporating risk scores based on levels of C-peptide and BMI significantly improved the predictability of PC-specific and all-cause mortality. The featured methods can be used to select an appropriate therapy for a subject with cancer, such as PC, and to treat a subject with cancer.

### Evaluating Risk of Mortality

The combination of a high energetic risk combined with an intermediate or high D'Amico risk is strongly correlated with mortality, e.g., mortality from PC, e.g., within about ten years of PC diagnosis. High energetic risk was defined as highest quartile levels of C-peptide and a BMI of 25-29.9 kg/m² or BMI≧30 kg/m². No such multi-marker approach for risk stratification has been proposed for subjects with PC or for evaluating subjects with no specific symptoms.

The 2010 NCCN guideline was adopted for the modified definition of D'Amico risk groups as follows: low-risk (PSA<10 ng/ml and Gleason 2-6 and clinical stage T1/T2); intermediate-risk (clinical stage T1/T2 with PSA 10-20 ng/ml or Gleason score 7); and high-risk (PSA>20 ng/ml, or Gleason score 8-10, or clinical stage T3).

### C-Peptide

C-peptide serves as a linker between the A- and the B-chains of insulin and facilitates the efficient assembly, folding, and processing of insulin in the endoplasmic reticulum. Equimolar amounts of C-peptide and insulin are then stored in the pancreatic beta cells, and both are released to the portal circulation. C-peptide has been used as a marker of insulin secretion and has been of great value in furthering the understanding of the pathophysiology of type 1 and type 2 diabetes. During the past decade, however, C-peptide has been found to be a bioactive peptide in its own right, with effects on microvascular blood flow and tissue health.

Three Variants of C-Peptide in Humans that Encode the Same Peptide

| Nucleic Acid | Amino Acid | GeneID |
|---|---|---|
| NM_000207.2 | NP_000198.1 | 3630 |
| NM_001185097.1 | NP_001172026.1 | 3630 |
| NM_001185098.1 | NP_001172027.1 | 3630 |

Unlike insulin, C-peptide is not appreciably cleared by the liver. Circulating C-peptide has a longer half-life (hours) than insulin (minutes) and serves as a good marker of insulin production. A consistent fraction of C-peptide production (5-10%) is excreted intact in the urine. Urinary clearance of C-peptide parallels the rate of production in healthy individuals. Urinary C-peptide is a novel noninvasive biomarker of insulin production, insulin sensitivity, and total energy intake in humans and animals.

Evaluating circulating levels of C-peptide in a subject typically includes obtaining a biological sample, e.g., serum, plasma, blood, or urine from the subject. Levels of C-peptide in the sample can be determined by measuring levels of peptide in the sample, using methods known in the art and/or described herein, e.g., immunoassays such as enzyme-linked immunosorbent assays (ELISA); for example, assay kits are commercially available from: antibodies-online; BACHEM; Calbiotech, Inc.; Dako; Diagnostic Systems Laboratories, Inc.; DRG International, Inc.; EMD Millipore; Mercodia AB; and Raybiotech, Inc. The level of C-peptide provides information regarding the subject's likelihood of experiencing an adverse outcome, e.g., mortality, e.g., within a specific time period, e.g., five years, six years, eight years, or ten years. The level of C-peptide can also provide information regarding the severity of disease in the subject.

### D'Amico Risk

D'Amico risk stratification is recommended by the National Comprehensive Cancer Network (NCCN) and the European Association of Urology (EAU) as clinical guideline for PC treatment options. D'Amico risk is based on (i) serum PSA levels; (ii) biopsy Gleason score (see, e.g., Gleason, D. F. (1977). “The Veteran's Administration Cooperative Urologic Research Group: histologic grading and clinical staging of prostatic carcinoma”. In Tannenbaum, M. Urologic Pathology: The Prostate. Philadelphia: Lea and Febiger. pp. 171-198; “Prostate Cancer: Diagnosis and Treatment,” National Collaborating Centre for Cancer (UK), Cardiff (UK): National Collaborating Centre for Cancer (UK); 2008 February (NICE Clinical Guidelines, No. 58)); and (iii) 1992 American Joint Commission on Cancer (AJCC) clinical tumor category (D'Amico et al. (JAMA 280:969-74, 1998).

Three possible D'Amico risk scores were assigned to each subject:

(a) a subject with a level of PSA less than 10 μg/mL, a Gleason score equal to or less than 6, and a clinical T1 or T2 stage has a low D'Amico risk;

(b) a subject with a level of 10-20 μg PSA/mL, a Gleason score of 7, or a clinical T1 or T2 stage has an intermediate D'Amico risk; and

(c) a subject with a level of greater than 20 μg PSA/mL, a Gleason score equal to or greater than 8, or a clinical T3 stage has a high D'Amico risk.

Methods and reagents are well known in the art for obtaining, processing, and analyzing samples (D'Amico et al. (JAMA 280:969-74, 1998).

### BMI

Obesity is a medical condition in which excess body fat has accumulated to the extent that it may have an adverse effect on health, leading to reduced life expectancy and/or increased health problems. BMI, a measurement that compares weight and height, defines people as overweight (pre-obese) when their BMI is between 25 kg/m² and 30 kg/m², and obese when it is greater than 30 kg/m².

Obesity increases the likelihood of various diseases, particularly certain types of cancer, Type 2 diabetes, heart disease, breathing difficulties during sleep, and osteoarthritis. Obesity is most commonly caused by a combination of excessive dietary calories, lack of physical activity, and genetic susceptibility, although some cases are caused primarily by genetic factors, endocrine disorders, medications, or psychiatric illness.

### General Methodology

In general, the methods described herein include determining mortality risk of a subject, e.g., a mammal, e.g., a human, by (1) evaluating circulating levels (e.g., levels in blood, serum, plasma, urine, or body tissue) of C-peptide; (2) determining BMI; and (3) determining D'Amico risk (e.g., one or more of PSA level, Gleason score, and clinical tumor stage).

In some embodiments, the risk of mortality is determined more than once; in that case, the higher measurement, or the most recent measurement, can be used. Thus, a risk of mortality can be determined that represents the change (e.g., the magnitude and direction, e.g., increase or decrease) in risk of mortality over time, e.g., over the course of a few days, e.g., three days or more, or over the course of weeks or months. Risk of mortality can also be determined multiple times to evaluate a subject's response to a treatment. For example, risk of mortality can be evaluated after administration of a treatment, e.g., one or more doses or rounds of a treatment, and compared to risk of mortality before the treatment was initiated. The difference between the risk of mortality levels would indicate whether the treatment was effective; e.g., a reduction in risk of mortality would indicate that the treatment was effective. The difference between the risk of mortality levels can also be used to monitor a subject's condition, e.g., to determine if the subject is improving, e.g., improving enough to be discharged from a hospital, to be treated less aggressively, or to be followed up at greater time intervals.

Once a level of risk of mortality has been determined, the level can be compared to a reference level. In some embodiments, the reference level will represent a threshold level, above which the subject has an increased risk of death, and/or has fatal PC. The reference level chosen may depend on the methodology used to measure the level of C-peptide, BMI, or D'Amico risk. For example, in some embodiments, where circulating levels of soluble C-peptide are determined using an immunoassay, and a level of C-peptide in the highest quartile of a reference group indicates that the subject has an increased risk of death, and/or has fatal PC.

In some embodiments, comparison of C-peptide to a reference level or ratio can be used to determine a subject's prognosis. For example, when the level of C-peptide is measured using an ELISA, the reference level can be used to determine prognosis as follows: a C-peptide level in the highest quartile of a reference group indicates that the subject has a poor prognosis, e.g., is not likely to recover; a C-peptide level in the lower three quartiles of a reference group indicates that the subject has a good prognosis, e.g., is likely to recover.

In some embodiments, D'Amico risk (e.g., PSA, stage, and grade) and BMI (e.g., physical measurements of body mass and height) can also be determined. Methods are well known in the art for determining D'Amico risk and BMI and the information from the comparison of three biomarkers with their respective reference levels provides cumulative information regarding an increased risk of death, and/or presence of a severe disease, e.g., PC, in the subject.

### Methods of Selecting an Appropriate Therapy

Methods of selecting an appropriate therapy for a subject with cancer, e.g., PC, include providing or obtaining a sample from a patient, and determining PC mortality risk in the patient. A sample can comprise serum, plasma, whole blood, or urine. The level of expression of C-peptide can be determined by immunohistochemistry.

If the level of C-peptide is at or above a reference level, e.g., in the highest quartile compared to the level of C-peptide in a reference group, it can be determined that a therapy comprising an anti-diabetic or insulin-lowering drug, e.g., metformin, is appropriate. If the level of C-peptide is below a reference level, e.g., in the lowest three quartiles compared to the level of C-peptide in a reference group, it can be determined that a therapy lacking an anti-diabetic or insulin-lowering drug is appropriate.

“Low” and “high” expression levels are relative values and are based on a comparison with those of a reference. In one embodiment, a reference level of expression is the expression level of C-peptide in a sample cancer population from which C-peptide expression data is collected. The expression level in a reference group can be determined by measuring C-peptide levels in the sample population. Typically, a subject exhibits “low” C-peptide levels if the level is less than the median C-peptide level in the reference, and a subject exhibits “high” C-peptide levels if the C-peptide level is above, or at or above, the highest quartile level in the reference. “Low” and “high” expression levels are relative and can be established with each new reference group. In one alternative, the expression level determined to be predictive of a subject's risk of mortality can be equal to or greater than the expression level of the highest third, or highest quartile of a reference, or the predictive expression level can be determined to be a level lower than the expression level of the lowest third, or lowest quartile of a reference.

The samples from a reference can be taken from subjects of the same species (e.g., human subjects). In some embodiments, the reference group can all have, for example, PC., e.g., fatal PC or non-fatal PC. The individual members of a reference may also share other similarities, such as similarities in stage of disease, previous treatment regimens, lifestyle (e.g., smokers or nonsmokers, overweight or underweight), or other demographics (e.g., age, genetic disposition). For example, besides having the same type of PC (e.g., fatal PC or non-fatal PC), patients in a reference may not have received any previous therapy. A reference should include expression analysis data from samples from at least 2, 5, 10, 15, 20, 25, 30, 40, 50, 60, 70, 80, 90, 100, 120, 140, 160, 180, or 200 subjects.

Expression levels in a reference can be determined by any method known in the art. Expression levels in a sample from a test subject are determined in the same manner as expression levels in the reference. For example, the level of C-peptide protein is detected. The presence and/or level of a protein can be evaluated using methods known in the art, e.g., using quantitative immunoassay methods such as enzyme linked immunosorbent assays (ELISAs), immunoprecipitations, immunofluorescence, immunohistochemistry, enzyme immunoassay (EIA), radioimmunoassay (RIA), diagnostic magnetic resonance, and Western blot analysis.

In some embodiments, high throughput methods, e.g., protein chips as are known in the art (see, e.g., Ch. 12, “Genomics,” in Griffiths et al., Eds. Modern Genetic Analysis, 1999, W. H. Freeman and Company; Ekins and Chu, Trends in Biotechnology, 1999; 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of C-protein.

A subject who has been determined by a method described herein to be at high risk of developing fatal prostate cancer can be administered a more aggressive therapy, whereas a subject determined to be at low risk of developing fatal prostate cancer can be treated more conservatively. Standard types of treatment include watchful waiting (for those at low risk); or surgery, radiation therapy, hormone therapy, chemotherapy, and/or biologic therapy.

Levels of C-peptide can be assayed and an appropriate therapy can be selected based on the observed expression levels. The therapy can include a single agent or multiple therapeutic agents (e.g., two, three, or more therapeutic agents). For example, when expression levels of C-peptide are determined to be high compared to a reference, a therapy comprising an anti-diabetic or insulin-lowering drug, e.g., metformin, can be selected. When expression levels of C-peptide are determined to be low compared to a reference, a therapy lacking an anti-diabetic or insulin-lowering drug can be selected.

Therapy can be administered to a subject using conventional dosing regimens. The appropriate dosage will depend on the particular therapeutic agents determined to be appropriate for the subject based on C-peptide expression levels as described herein.

Therapy can be administered by standard methods, including orally, such as in the form of a pill, intravenously, rectally, by injection into a body cavity, intraperitoneally, intramuscularly, or intrathecally. A therapy regimen can be delivered as a continuous regimen, e.g., rectally, intravenously, orally, or in a body cavity. A therapy regimen can be delivered in a cycle including the day or days the drug is administered followed by a rest and recovery period. The recovery period can last for one, two, three, or four weeks or more, and then the cycle can be repeated. A course of therapy can include at least two to 12 cycles (e.g., three, four, five, six, seven, ten or twelve cycles).

Upon administration of a therapy according to the risk of mortality, a patient can be monitored for a response to the therapy. For example, risk of mortality can be evaluated before and after administration of the therapy to monitor disease progression. If risk of mortality decreases, the disease can be determined to be in remission, or regressing towards remission. A partial decrease in risk of mortality can indicate a disease in partial remission, and if the risk of mortality completely disappears, the disease can be said to be in complete remission. If risk of mortality increases, the disease can be determined to be progressing. If risk of mortality does not change following administration of the therapy, the disease can be categorized as stable.

A subject can also be assessed according to his physical condition, with attention to factors such as weight loss and other symptoms related to the cancer. For example, symptoms of PC, include trouble urinating, decreased force in the stream of urine, blood in urine, blood in semen, swelling in legs, discomfort in the pelvic area, and bone pain.

The invention is further illustrated by the following examples, which should not be construed as further limiting.

### EXAMPLES

Data from the two large long-term prospective cohorts of men with localized PC demonstrate strong associations of the energetic risk with PC-specific and all-cause mortality among men with localized disease. Adding energetic risk to D'Amico risk significantly improved the prediction of these two major outcomes.

### Example 1

### Study Populations

The analysis includes PC cases from the Physicians' Health Study (PHS) and the Health Professionals Follow-up Study (HPFS). The PHS was a randomized trial of aspirin and beta carotene among 22,071 U.S. male physicians, aged 40-84 in 1982, without a history of heart disease, cancer, or other major chronic diseases. Among the 14,916 PHS participants who provided blood samples during 1982-1984, plasma C-peptide data were available for 843 men diagnosed with incident PC during follow-up. Excluded were 55 (6.5%) men with regional or distant metastasis (T4/N1/M1) at diagnosis, 88 (10%) with missing clinical information, and 9 (1.1%) with a history of diabetes before blood collection, leaving 691 men with localized PC in the final analysis.

The HPFS is a prospective cohort study of 51,529 men established in 1986. Among the 18,018 HPFS participants who provided a blood specimen during 1993-1995, 1,331 incident PC cases were confirmed and plasma C-peptide data were available for 1,317 cases. Similarly, 32 (2.4%) men were excluded with regional or distant metastatis (T4/N1/M1) at diagnosis, 112 (7.9%) with missing clinical information, and 62 (5.3%) with a history of diabetes before blood collection, leaving 1,111 men with localized PC in the final analysis.

In both cohorts, BMI (kg/m²), cigarette smoking status, and time since last meal at time of blood draw, was assessed as well as incident diabetes after blood draw.

Confirmation of PC, Outcome Follow-Up, and Definition of Modified D'Amico Risk

Self-reported cases of PC from the follow-up questionnaires were confirmed by medical records and pathology reports; only confirmed cases were included in the analysis. PC stage was recorded according to the TNM staging system or converted from a modified Whitmore-Jewett classification scheme (for cases diagnosed during the early years of follow-up). PC cases with T1a disease (i.e., incidental microscopic focal tumors) in the HPFS were excluded. We recorded PSA concentrations at diagnosis from medical records for all HPFS cases. However, among the 691 men in the PHS, 170 men were diagnosed either before PSA was clinically available or had missing diagnostic PSA; we therefore used the levels of PSA measured at baseline, in blood samples drawn in 1982 (Gann et al., JAMA 273:289-94, 1995) as a surrogate. The 2010 NCCN guideline was adopted for the modified definition of D'Amico risk groups as follows: low-risk (PSA<10 ng/ml and Gleason 2-6 and clinical stage T1/T2); intermediate-risk (clinical stage T1/T2 with PSA 10-20 ng/ml or Gleason score 7); and high-risk (PSA>20 ng/ml, or Gleason score 8-10, or clinical stage T3). Deaths were ascertained through searching the National Death Index. Follow-up for all fatal outcomes was virtually complete. Cohorts for mortality were routinely followed through repeated mailings, telephone calls to non-respondents, and periodic searches of the National Death Index. All deaths are confirmed through extensive review of death certificates and medical records by an Endpoints Committee of four physicians including Drs. Stampfer and Giovannucci.

PSA and C-Peptide Assay

Prediagnostic plasma was frozen at −80 or −167° C. up to the time of assay measurement. The methods for baseline PSA measurement (Gann et al., JAMA 273:289-94, 1995) in PHS and C-peptide in both cohorts have been reported elsewhere (Ma et al., Lancet Oncol. 9:1039-47, 2008). Specifically, C-peptide concentrations were measured in blood that had been frozen at −82° C., by use of standard ELISA and a single production lot of reagents (Diagnostic Systems Limited, Webster, Tex., USA). Blinded embedded quality control samples showed a within-assay CV<5% and a between-assay variability <9%.

Statistical Analysis

All analyses were conducted in the same fashion for PHS (discovery set) and HPFS (validation set) separately, and then jointly. Stratified Cox regression models (Andersen et al., Statistical Models Based on Counting Processes, New York: Springer-Verlag; 1993) were used to estimate the combined hazard ratios (HR) and 95% confidence intervals (95% CI) for risk of death from PC, other causes and any cause while adjusting for study, according to the following predictors: D'Amico risk group, prediagnostic BMI (<25, 25-29.9, ≧30 kg/m²), C-peptide (cohort- and fasting-specific cut-off quartiles), different combinations of BMI and C-peptide, and the “energetic risk” (BMI≧25 kg/m² and high C-peptide; or BMI≧30 kg/m²). D'Amico risk was also categorized into six groups according to high vs. low BMI (≧25 vs. <25 kg/m²), C-peptide (highest quartile vs. the rest), or energetic risk (high vs. low). The D'Amico low-risk and low energetic risk group was used as the common referent group. Person-years were counted from date of PC diagnosis (time 0) to date of PC death (event), death from other causes, or the end of follow-up (Mar. 9, 2010 in the PHS or February 2011 in the HPFS), whichever came first. All models included age at diagnosis, smoking status at blood draw, diabetes after blood draw, assay batches (for C-peptide related analysis), and time between blood draw and PC diagnosis. SAS (version 9.1.3; SAS Institute Inc., Cary, N.C.) was used applying a two-sided significance level of 0.05. STATA (version 11.0) was used to estimate and compare the concordance index (c-statistic) (Harrell et al., JAMA 247:2543-6, 1982) to compare the predictability of the modified D'Amico risk model with the model incorporating energetic risk together with the modified D'Amico risk.

### Example 2

The characteristics of men in the two cohorts are provided in Table 1. In both cohorts, those who died of PC tended to die at younger age (˜78 years) than men who died of other causes (81-83 years), had higher PSA (≧20 ng/ml), stage (T3), grade tumors (Gleason 7-10), and a higher D'Amico risk category. Those who died tended to be smokers, had higher C-peptide levels, and higher energetic risk.

In the PHS, the associations for PC-specific mortality were first compared using D'Amico risk created by baseline PSA (68 fatal PC, 377 censored) with that created by PSA at diagnosis (53 fatal PC, 306 censored) and the hazard ratios (HRs; 95% CI) for D'Amico low-risk, intermediate-risk and high-risk were 1.0, 3.3 (1.7-6.2), and 6.4 (3.4-12.1) using baseline PSA and 1.0, 3.0 (1.1-8.6), and 7.1 (2.6-19.4) using PSA at diagnosis. These data support the validity of using prediagnostic PSA as a surrogate for defining the D'Amico risk for men who had missing diagnostic PSA.

Next, the risk for death from PC and death from other causes was evaluated according to the modified D'Amico risk group alone, or in combination with BMI, C-peptide or different combination of BMI and C-peptide, controlling for age at diagnosis, diabetes after blood draw, baseline smoking status, and year from baseline to PC diagnosis (Table 2). Since results were similar in PHS (discovery) and in HPFS (validation), the two cohorts were combined in the following analyses. Men with high-risk disease had a significant 4.7-times higher risk of PC-specific mortality compared to those with D'Amico low-risk cancer. In the multivariate models controlling for the D'Amico risk, overweight (BMI 25-29.9 kg/m²) and obesity (BMI≧30 kg/m²) were significantly predictive for PC-specific mortality. Replacing BMI with C-peptide levels in the same model showed a nonlinear association in both cohorts with an elevated risk, as apparent in the highest quartile (combined HR_{Q4 vs. Q1-3}=1.8; 95% CI: 1.1-3.0). After evaluating different combinations of BMI and C-peptide (models 5 and 6), the most consistent risk estimates in both cohorts were seen when combining BMI 25-29.9 kg/m² and C-peptide in the highest quartile together with BMI≧30 kg/m² as a high “energetic risk” (HR_{high vs. low}=2.8; 95% CI: 1.6-5.1; Table 2). In all the models, the risk estimate for D'Amico risk did not materially change.

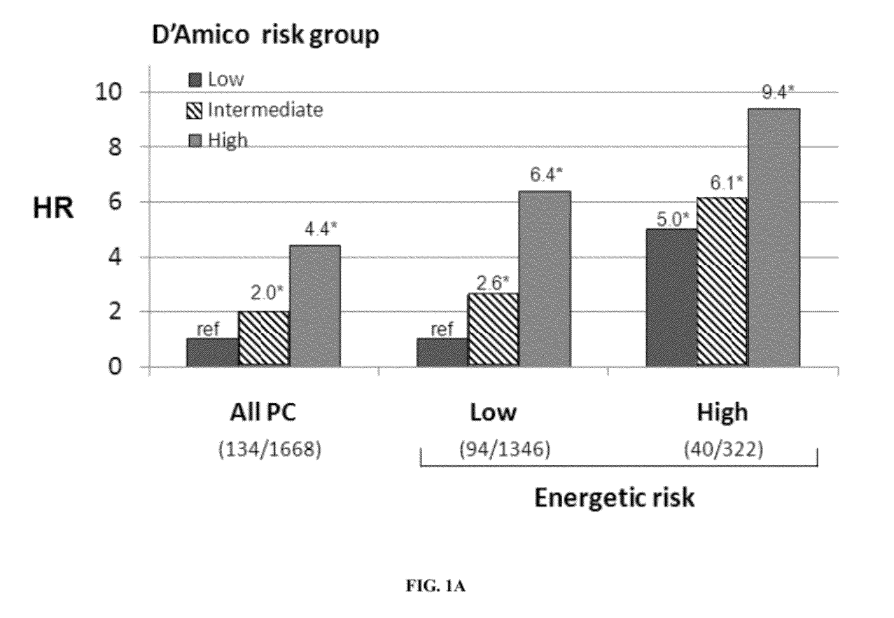

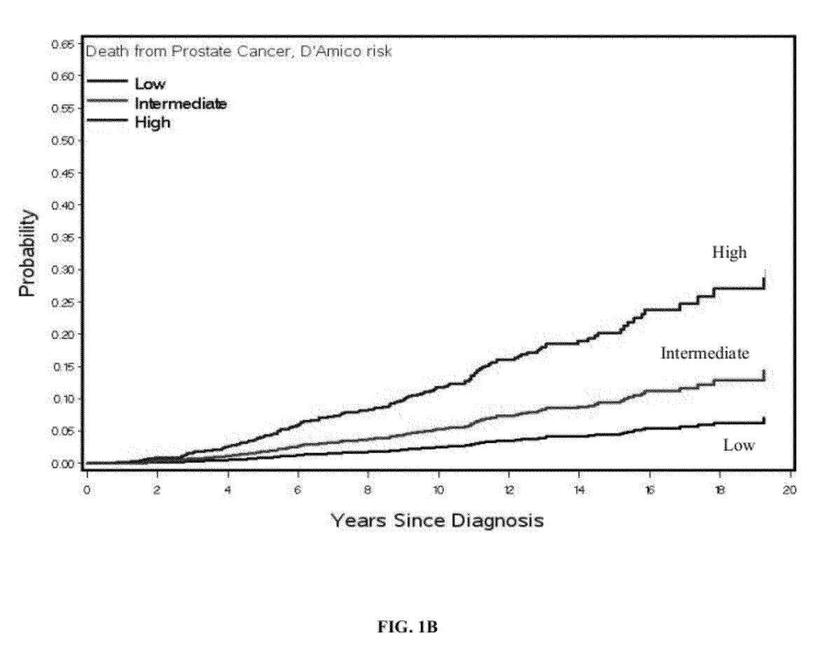

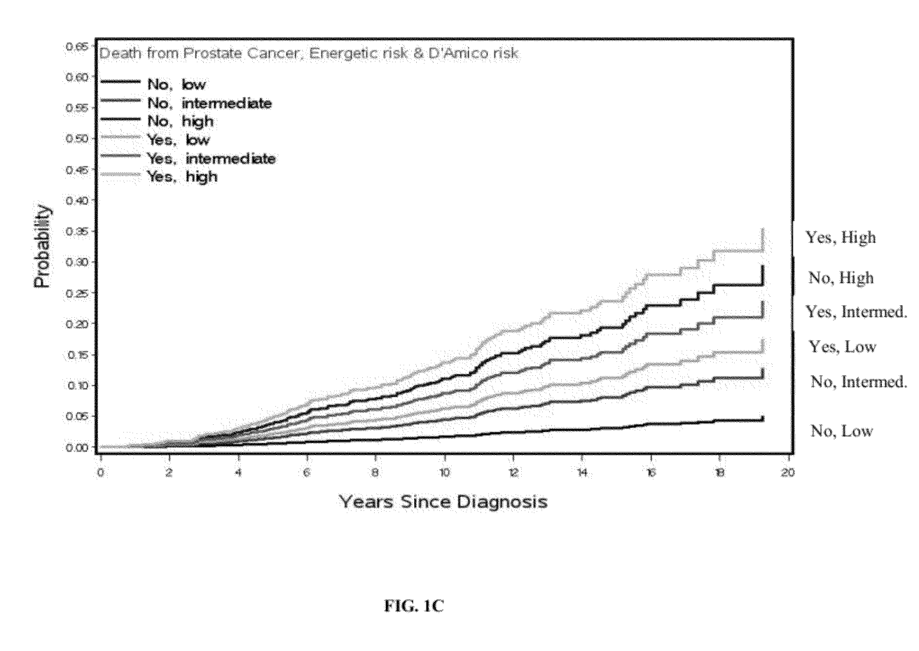

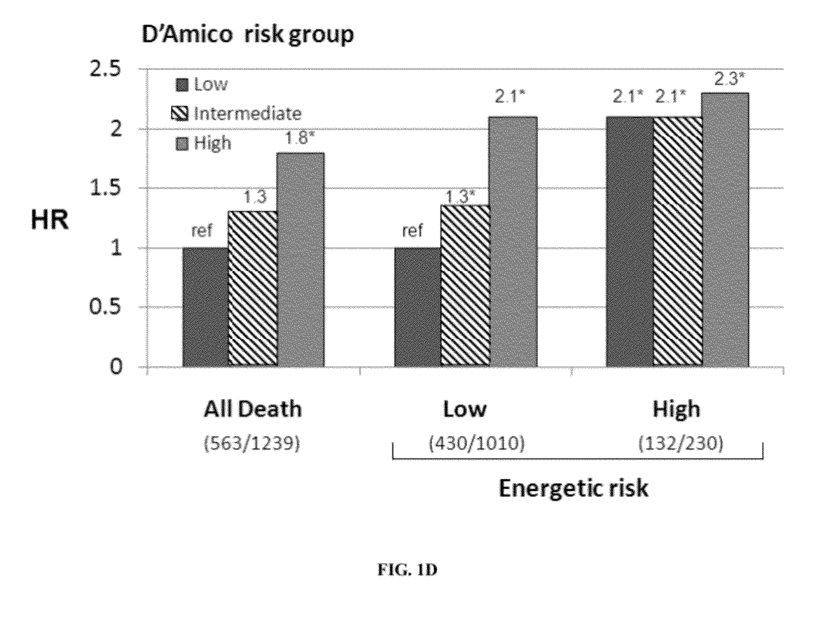

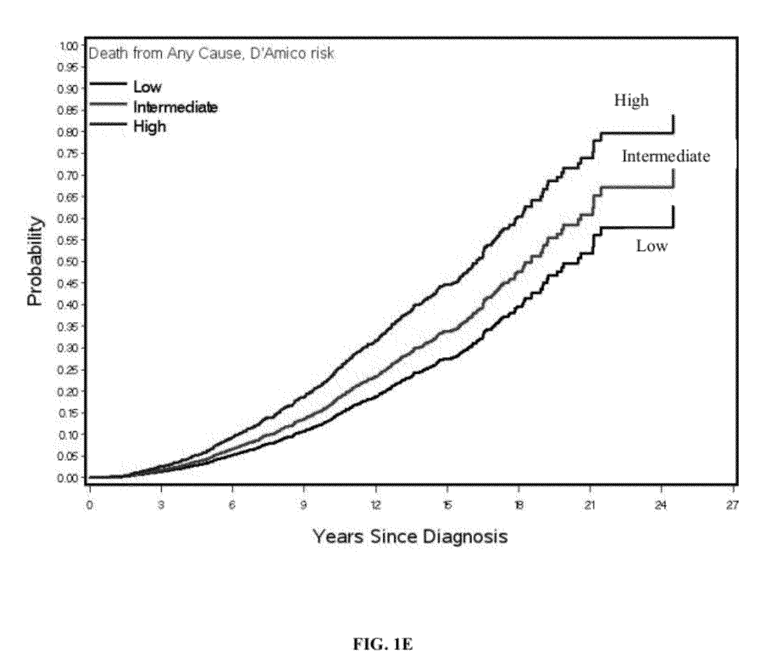

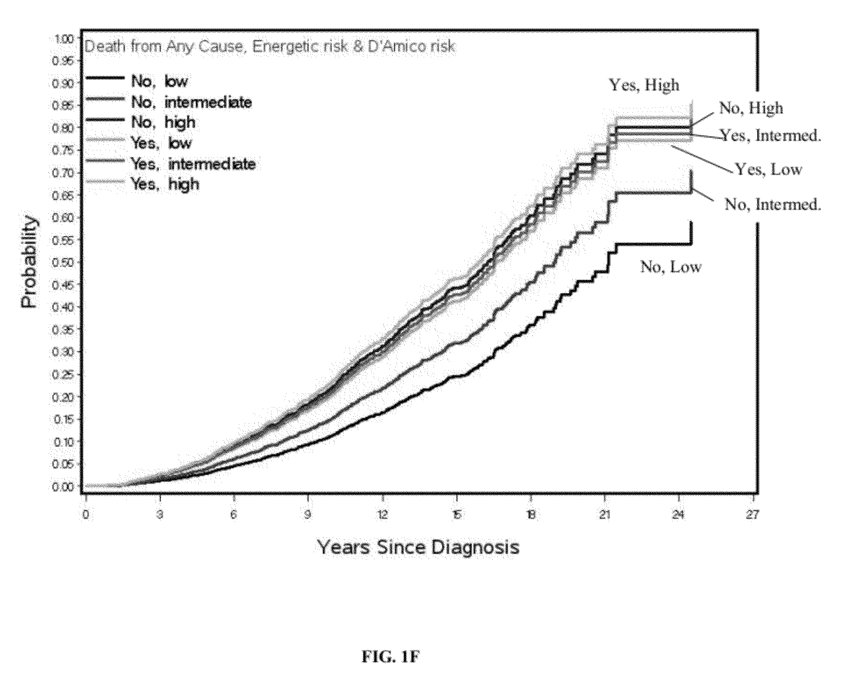

To assess whether incorporating the metabolic risk factors into the modified D'Amico risk group could improve the prognosis for PC-specific mortality, the three D'Amico risk categories were regrouped according to high vs. low BMI, C-peptide, or energetic risk (Table 3). Adding the energetic risk score provided the best improvement; a high energetic risk reclassifed 20% men in each of the three D'Amico risk groups to a higher risk of PC-specific mortality than those with low energetic risk (FIGS. 1A-C). Again, results were similar in the two cohorts (Table 4).

In these men with localized PC, most (74% in the PHS and 79% in the HPFS) of the deaths were from causes other than PC (Table 1). Obesity was a significant predictor of death from other causes, but elevated C-peptide predicted death from other causes both among lean (BMI<25 kg/m²) and overweight men (BMI≧25 kg/m², Table 2, model 6). Men with high energetic risk (˜20% of the study population) had a similar risk of all-cause mortality equivalent to a D'Amico high-risk category in men with a low energetic risk (Table 3, FIGS. 1D-F).

Finally, the performance of different predictors for PC-specific mortality and all-cause mortality was compared using the C-statistics (Table 5). Incorporating the energetic risk into the D'Amico risk significantly improved the predictability for PC-specific mortality (C-statistic changed from 0.72 to 0.78, P<0.0001), with a larger improvement observed in men with low-intermediate risk (C-statistic from 0.66 to 0.74; P=0.048). The improvement for all-cause mortality was relatively small but statistically significant.

TABLE 1
Characteristics of men initially diagnosed with localized prostate cancer (PC) in the
Physicians' Health Study (PHS) and the Health Professionals Follow-up Study (HPFS)

| | PHS (n = 691) | | | | | | HPFS (n = 1111) | | | | | |
|---|---|---|---|---|---|---|---|---|---|---|---|---|
| | (1982-2005) | | | | | | (1993-2004) | | | | | |
| | PC death | | Other death | | Survivors | | PC death | | Other death | | Survivors | |
| No. (%) | 78 | (11.3) | 223 | (32.3) | 390 | (56.4) | 56 | (5.04) | 206 | (18.54) | 849 | (76.42) |
| Age at study entry | 62.1 ± 7.5 | | 63.2 ± 7.3 | | 55.0 ± 6.5 | | 67.1 ± 8.4 | | 68.9 ± 6.3 | | 62.0 ± 7.2 | |
| Age at diagnosis (yr) | 70.1 ± 7.5 | | 71.9 ± 6.6 | | 67.1 ± 7.0 | | 70.8 ± 7.6 | | 73.2 ± 6.0 | | 67.7 ± 7.2 | |
| Age at death (yr) | 78.8 ± 8.0 | | 83.1 ± 7.2 | | | | 77.9 ± 7.2 | | 81.3 ± 6.7 | | | |
| Duration from baseline to | 8.3 | (0.0-17.9) | 8.9 | (0.1-21.9) | 11.8 | (0.3-23.5) | 3.6 | (0.1-9.9) | 4.2 | (0.1-10.1) | 6.0 | (0.1-10.6) |
| PC (yr) | | | | | | | | | | | | |
| Duration from PC diagnosis | 8.6 | (1.2-19.2) | 11.0 | (0.0-24.5) | 15.4 | (3.9-27.2) | 6.7 | (0.8-15.2) | 8.0 | (1.4-16.1) | 11.1 | (7.1-17.6) |
| to death/censored (yr) | | | | | | | | | | | | |
| PSA at diagnosis,¹ No (%) | | | | | | | | | | | | |
| 0-<4 | 25 | (32.9) | 52 | (23.9) | 76 | (19.7) | 3 | (6.00) | 18 | (9.14) | 104 | (12.32) |
| 4-<10 | 16 | (21.1) | 72 | (33.0) | 197 | (51.0) | 25 | (50.00) | 101 | (51.27) | 557 | (66.00) |
| 10-<=20 | 17 | (22.4) | 55 | (25.2) | 85 | (22.0) | 10 | (20.00) | 60 | (30.46) | 140 | (16.59) |
| >20 | 18 | (23.7) | 39 | (17.9) | 28 | (7.3) | 12 | (24.00) | 18 | (9.14) | 43 | (5.09) |
| Unknown | 2 | | 5 | | 4 | | 6 | | 9 | | 5 | |
| Clinical stage | | | | | | | | | | | | |
| T1/T2 | 68 | (87.2) | 208 | (93.3) | 372 | (95.4) | 43 | (76.79) | 186 | (90.29) | 753 | (88.69) |
| T3 | 10 | (12.8) | 15 | (6.7) | 18 | (4.6) | 13 | (23.21) | 20 | (9.71) | 96 | (11.31) |
| Gleason sum | | | | | | | | | | | | |
| 2-6 | 28 | (37.8) | 140 | (64.2) | 280 | (71.8) | 16 | (29.63) | 120 | (59.41) | 544 | (64.38) |
| 7 | 25 | (33.8) | 59 | (27.1) | 83 | (21.3) | 22 | (40.74) | 53 | (26.24) | 243 | (28.76) |
| 8-10 | 21 | (28.4) | 19 | (8.7) | 27 | (6.9) | 16 | (29.63) | 29 | (14.36) | 58 | (6.86) |
| Unknown | 4 | | 5 | | 0 | | 2 | | 4 | | 4 | |
| D'Amico risk group² | | | | | | | | | | | | |
| Low | 14 | (18.0) | 88 | (39.5) | 203 | (52.1) | 13 | (23.21) | 75 | (36.41) | 412 | (48.53) |
| Intermediate | 28 | (35.9) | 73 | (32.7) | 126 | (32.3) | 16 | (28.57) | 80 | (38.83) | 272 | (32.04) |
| High | 36 | (46.2) | 62 | (27.8) | 61 | (15.6) | 27 | (48.21) | 51 | (24.76) | 165 | (19.43) |
| Diabetes after blood draw | | | | | | | | | | | | |
| No | 76 | (97.4) | 206 | (92.4) | 341 | (87.4) | 52 | (92.86) | 179 | (86.89) | 769 | (90.58) |
| Yes | 2 | (2.6) | 17 | (7.6) | 49 | (12.6) | 4 | (7.14) | 27 | (13.11) | 80 | (9.42) |
| Smoking status⁴ | | | | | | | | | | | | |
| Never | 40 | (51.3) | 86 | (38.6) | 206 | (52.8) | 25 | (44.64) | 81 | (39.32) | 472 | (55.59) |
| Ever | 38 | (48.7) | 137 | (61.4) | 184 | (47.2) | 31 | (55.36) | 125 | (60.68) | 377 | (44.41) |
| BMI (kg/m²) | 25.1 ± 2.6 | | 24.7 ± 2.2 | | 24.5 ± 2.4 | | 25.8 ± 3.3 | | 25.9 ± 3.5 | | 25.6 ± 3.1 | |
| C-peptide, ng/ml, (25^{th}-75^{th}) | 2.2 | (0.9-3.3) | 1.8 | (1.2-3.0) | 1.5 | (0.9-2.5) | 1.9 | (1.4-2.7) | 2.0 | (1.4-3.7) | 1.8 | (1.3-2.8) |
| Energetic risk³ | | | | | | | | | | | | |
| Low | 58 | (74.4) | 185 | (83.0) | 338 | (86.7) | 36 | (64.29) | 152 | (73.79) | 671 | (79.03) |
| High | 20 | (25.6) | 38 | (17.0) | 52 | (13.3) | 20 | (35.71) | 54 | (26.21) | 178 | (20.97) |

¹Baseline PSA levels were used as surrogate of PSA at diagnosis for 170 men in the PHS.
²D'Amico risk group: low risk: PSA <10 ng/ml, Gleason ≦6 and clinical stage T1/T2; intermediate risk: PSA 10-20 ng/ml or Gleason = 7; high risk: PSA >20 ng/ml, Gleason ≧8, or clinical stage T3 at diagnosis.
³Energetic risk: BMI ≧30 kg/m² or BMI 25-29.9 kg/m² and C-peptide in the highest quartile.
⁴Baseline smoking status

TABLE 2
Adjusted hazard ratio (HR) and 95% confidence interval (95% CI) of prostate cancer-specific mortality
and other cause of death according to modified D'Amico risk score and BMI, C-peptide levels,
or the combination of BMI and C-peptide in the two cohorts of men with localized disease

| Discovery Set | | Validation Set | | | |
|---|---|---|---|---|---|
| PHS (1982-2010) | | HPFS (1993-2010) | | Combined Sets | |
| Death/ | HR | Death/ | HR | Death/ | HR |
| censored | (95% CI) | censored | (95% CI) | censored | (95% CI) |

PC Death
Model 1 ¹

| | | | | | | |
|---|---|---|---|---|---|---|
| Age | 78/613 | 1.05 (1.01-1.09) | 56/1055 | 1.05 (1.01-1.10) | 134/1668 | 1.05 (1.03-1.08) |
| D'Amico risk | | | | | | |
| Low | 14/291 | 1.00 (ref) | 13/487 | 1.00 (ref) | 27/778 | 1.00 (ref) |
| Intermediate | 28/199 | 2.44 (1.28-4.65) | 16/352 | 1.57 (0.76-3.28) | 44/551 | 2.02 (1.25-3.26) |
| High | 36/123 | 4.74 (2.54-8.84) | 27/216 | 4.03 (2.07-7.87 | 63/339 | 4.43 (2.81-6.98) |

Model 2 ²

| | | | | | | |
|---|---|---|---|---|---|---|
| BMI (kg/m²) | | | | | | |
| <25 | 37/379 | 1.00 (ref) | 23/467 | 1.00 (ref) | 60/846 | 1.00 (ref) |
| 25-29.9 | 39/218 | 1.75 (1.11-2.76) | 25/495 | 1.13 (0.64-2.00) | 64/713 | 1.45 (1.02-2.08) |
| ≧30 | 2/16 | 1.07 (0.25-4.50) | 8/93 | 2.52 (1.09-5.81) | 10/109 | 2.08 (1.05-4.13) |

Model 3 ³

| | | | | | | |
|---|---|---|---|---|---|---|
| C-peptide | | | | | | |
| Q1 | 19/153 | 1.00 (ref) | 12/265 | 1.00 (ref) | 31/418 | 1.00 (ref) |
| Q2 | 14/159 | 0.91 (0.46-1.83) | 12/266 | 0.95 (0.43-2.13) | 26/425 | 0.92 (0.55-1.56) |
| Q3 | 19/154 | 1.26 (0.66-2.38) | 15/264 | 1.10 (0.51-2.38) | 34/418 | 1.20 (0.73-1.95) |
| Q4 | 26/147 | 1.93 (1.05-3.54) | 17/260 | 1.61 (0.75-3.46) | 43/407 | 1.76 (1.10-2.82) |

Model 4 ³

| | | | | | | |
|---|---|---|---|---|---|---|
| C-peptide Q1-3 | 52/466 | 1.00 (ref) | 39/795 | 1.00 (ref) | 91/1261 | 1.00 (ref) |
| C-peptide Q4 | 26/147 | 1.84 (1.14-2.98) | 17/260 | 1.58 (0.88-2.83) | 43/407 | 1.70 (1.17-2.45) |

Model 5 ³

| | | | | | | |
|---|---|---|---|---|---|---|
| BMI/C-peptide ⁴ | | | | | | |
| Low/low | 30/314 | 1.00 (ref) | 22/402 | 1.00 (ref) | 52/716 | 1.00 (ref) |
| Low/high | 7/65 | 1.20 (0.53-2.76) | 1/65 | 0.36 (0.05-2.68) | 8/130 | 0.89 (0.42-1.88) |
| High/low | 22/152 | 1.34 (0.77-2.34) | 17/393 | 0.90 (0.48-1.70) | 39/545 | 1.15 (0.76-1.75) |
| High/high | 19/82 | 2.84 (1.57-5.14) | 16/195 | 1.89 (0.97-3.68) | 35/277 | 2.35 (1.52-3.65) |

Model 6 ³

| | | | | | | |
|---|---|---|---|---|---|---|
| BMI/C-peptide ⁵ | | | | | | |
| Low/low | 30/314 | 1.00 (ref) | 22/402 | 1.00 (ref) | 52/716 | 1.00 (ref) |
| Low/high | 7/65 | 1.20 (0.52-2.75) | 1/65 | 0.36 (0.05-2.67) | 8/130 | 0.89 (0.42-1.88) |
| Medium/low | 21/144 | 1.38 (0.79-2.42) | 13/356 | 0.75 (0.38-1.50) | 34/500 | 1.09 (0.70-1.68) |
| Medium/high | 18/74 | 2.96 (1.61-5.43) | 12/139 | 1.86 (0.91-3.81) | 30/213 | 2.36 (1.49-3.74) |
| BMI ≧30 kg/m² | 2/16 | 1.14 (0.27-4.85) | 8/93 | 2.36 (1.02-5.47) | 10/109 | 2.07 (1.04-4.12) |
| Energetic risk ^{3, 6} | | | | | | |
| Low | 58/523 | 1.00 (ref) | 36/823 | 1.00 (ref) | 94/1346 | 1.00 (ref) |
| High | 20/90 | 2.22 (1.32-3.74) | 20/232 | 2.38 (1.36-4.18) | 40/322 | 2.24 (1.53-3.26) |

Other Death
Model 1 ¹

| | | | | | | |
|---|---|---|---|---|---|---|
| Age | 223/468 | 1.12 (1.10-1.15) | 206/905 | 1.13 (1.10-1.16) | 429/1373 | 1.13 (1.11-1.14) |
| D'Amico risk | | | | | | |
| Low | 88/217 | 1.00 (ref) | 75/425 | 1.00 (ref) | 163/642 | 1.00 (ref) |
| Intermediate | 73/154 | 0.99 (0.72-1.35) | 80/288 | 1.28 (0.93-1.76) | 153/442 | 1.13 (0.91-1.41) |
| High | 62/97 | 1.41 (1.02-1.96) | 51/192 | 1.27 (0.88-1.81) | 113/289 | 1.34 (1.05-1.71) |

Model 2 ²

| | | | | | | |
|---|---|---|---|---|---|---|
| BMI (kg/m²) | | | | | | |
| <25 | 129/287 | 1.00 (ref) | 89/401 | 1.00 (ref) | 218/688 | 1.00 (ref) |
| 25-29.9 | 90/167 | 1.21 (0.92-1.59) | 93/427 | 1.00 (0.74-1.35) | 183/594 | 1.09 (0.89-1.33) |
| ≧30 | 4/14 | 0.68 (0.25-1.85) | 24/77 | 1.76 (1.10-2.81) | 28/91 | 1.50 (1.00-2.24) |

Model 3 ³

| | | | | | | |
|---|---|---|---|---|---|---|
| C-peptide | | | | | | |
| Q1 | 50/122 | 1.00 (ref) | 35/242 | 1.00 (ref) | 85/364 | 1.00 (ref) |
| Q2 | 57/116 | 1.30 (0.89-1.91) | 52/226 | 1.45 (0.94-2.24) | 109/342 | 1.34 (1.01-1.78) |
| Q3 | 47/126 | 0.99 (0.66-1.48) | 54/225 | 1.37 (0.89-2.12) | 101/351 | 1.15 (0.86-1.54) |
| Q4 | 69/104 | 1.85 (1.27-2.69) | 65/212 | 1.98 (1.29-3.04) | 134/316 | 1.89 (1.43-2.50) |

Model 4 ³

| | | | | | | |
|---|---|---|---|---|---|---|
| C-peptide Q1-3 | 154/364 | 1.00 (ref) | 141/693 | 1.00 (ref) | 295/1057 | 1.00 (ref) |
| C-peptide Q4 | 69/104 | 1.69 (1.26-2.26) | 65/212 | 1.54 (1.13-2.09) | 134/316 | 1.62 (1.31-2.00) |

Model 5 ³

| | | | | | | |
|---|---|---|---|---|---|---|
| BMI/C-peptide ⁴ | | | | | | |
| Low/low | 96/248 | 1.00 (ref) | 71/353 | 1.00 (ref) | 167/601 | 1.00 (ref) |
| Low/high | 33/39 | 1.78 (1.19-2.66) | 18/48 | 1.77 (1.05-2.99) | 51/87 | 1.79 (1.30-2.46) |
| High/low | 58/116 | 1.15 (0.83-1.60) | 70/340 | 1.06 (0.76-1.48) | 128/456 | 1.10 (0.87-1.39) |
| High/high | 36/65 | 1.78 (1.20-2.64) | 47/164 | 1.52 (1.03-2.24) | 83/229 | 1.63 (1.24-2.15) |

Model 6 ³

| | | | | | | |
|---|---|---|---|---|---|---|
| BMI/C-peptide ⁵ | | | | | | |
| Low/low | 96/248 | 1.00 (ref) | 71/353 | 1.00 (ref) | 167/601 | 1.00 (ref) |
| Low/high | 33/39 | 1.78 (1.19-2.66) | 18/48 | 1.77 (1.05-2.99) | 51/87 | 1.79 (1.30-2.46) |
| medium/low | 56/109 | 1.20 (0.86-1.67) | 63/306 | 1.05 (0.74-1.48) | 119/415 | 1.11 (0.88-1.41) |
| medium/high | 34/58 | 1.79 (1.19-2.68) | 30/121 | 1.24 (0.80-1.92) | 64/179 | 1.47 (1.10-1.98) |
| BMI ≧30 kg/m² | 4/14 | 0.79 (0.29-2.16) | 24/77 | 1.96 (1.21-3.17) | 28/91 | 1.70 (1.13-2.57) |
| Energetic risk ^{3, 6} | | | | | | |
| Low | 185/396 | 1.00 (ref) | 152/707 | 1.00 (ref) | 337/1103 | 1.00 (ref) |
| High | 38/72 | 1.36 (0.95-1.95) | 54/198 | 1.37 (0.99-1.89) | 92/270 | 1.37 (1.08-1.74) |

¹ Multivariate model controlled for age at diagnosis, diabetes after blood draw, baseline smoking status, and year from baseline to PCa diagnosis;
² Also controlled for D'Amico risk group;
³ Also controlled for D'Amico risk group and C-peptide assay batches; C-peptide quartile cut-points were based on fasting status (i.e., hour since last meal <4 hr vs. ≧4 hr);
⁴ Low/low: BMI <25 kg/m² and C-peptide in quartile 1-3, low/high: BMI <25 kg/m² and C-peptide in quartile 4, high/low: BMI ≧25 kg/m² and C-peptide in quartile 1-3, high/high: BMI ≧25 kg/m² and C-peptide in quartile 4;
⁵ Low/low: BMI <25 kg/m² and C-peptide in quartile 1-3, low/high: BMI <25 kg/m² and C-peptide in quartile 4, medium/low: BMI ≧25-<30 kg/m² and C-peptide in quartile 1-3, medium/high: BMI ≧25-<30 kg/m² and C-peptide in quartile 4;
⁶ In the same mode, energetic risk: BMI ≧25-<30 kg/m² AND C-peptide in quartile 4 OR BMI ≧30 kg/m²; diabetes diagnosed after blood draw.

TABLE 3
Adjusted hazard ratio (HR)¹ and 95% confidence interval (95% CI) of death from prostate cancer, other
cause, and any cause combining data from the two cohorts of men with localized prostate cancer

| | | Death from PC | | Death from Other Cause | | Death from Any Cause | |
|---|---|---|---|---|---|---|---|
| Energetic | | Fatal/ | HR | Fatal/ | HR | Fatal/ | HR |
| Parameter | D'Amico risk | censored | (95% CI) | censored | (95% CI) | censored | (95% CI) |
| BMI | | | | | | | |
| <25 kg/m² | Low | 10/396 | 1.00 (ref) | 84/322 | 1.00 (ref) | 94/312 | 1.00 (ref) |
| | Intermediate | 23/284 | 3.01 (1.43-6.34) | 74/233 | 1.12 (0.82-1.53) | 97/210 | 1.32 (0.99-1.75) |
| | High | 27/166 | 5.47 (2.64-11.3) | 60/133 | 1.45 (1.04-2.03) | 87/106 | 1.89 (1.41-2.54) |
| ≧25 kg/m² | Low | 17/382 | 2.35 (1.07-5.15) | 79/320 | 1.17 (0.85-1.60) | 96/303 | 1.30 (0.97-1.74) |
| | Intermediate | 21/267 | 3.32 (1.56-7.08) | 79/209 | 1.32 (0.97-1.81) | 100/188 | 1.54 (1.16-2.04) |
| | High | 36/173 | 8.61 (4.25-17.4) | 53/156 | 1.43 (1.01-2.03) | 89/120 | 2.18 (1.63-2.93) |
| C-peptide | | | | | | | |
| Q1-3 | Low | 15/583 | 1.00 (ref) | 105/493 | 1.00 (ref) | 120/478 | 1.00 (ref) |
| | Intermediate | 28/435 | 2.25 (1.20-4.22) | 114/349 | 1.28 (0.98-1.67) | 142/321 | 1.40 (1.10-1.79) |
| | High | 48/243 | 6.35 (3.55-11.4) | 76/215 | 1.46 (1.08-1.96) | 124/167 | 2.09 (1.62-2.69) |
| Q4 | Low | 12/195 | 2.99 (1.39-6.40) | 58/149 | 1.99 (1.43-2.75) | 70/137 | 2.11 (1.57-2.85) |
| | Intermediate | 16/116 | 5.55 (2.73-11.3) | 39/93 | 1.72 (1.19-2.50) | 55/77 | 2.18 (1.58-3.00) |
| | High | 15/96 | 6.21 (3.02-12.8) | 37/74 | 2.21 (1.51-3.22) | 52/59 | 2.68 (1.93-3.73) |
| Energetic risk | | | | | | | |
| Low | Low | 15/629 | 1.00 (ref) | 124/520 | 1.00 (ref) | 139/505 | 1.00 (ref) |
| | Intermediate | 31/450 | 2.60 (1.40-4.82) | 121/360 | 1.19 (0.93-1.53) | 152/329 | 1.34 (1.07-1.69) |
| | High | 48/267 | 6.38 (3.56-11.4) | 92/223 | 1.51 (1.15-1.99) | 140/175 | 2.05 (1.62-2.60) |
| High | Low | 12/149 | 5.04 (2.35-10.8) | 39/122 | 1.78 (1.23-2.58) | 51/110 | 2.14 (1.54-2.97) |
| | Intermediate | 13/101 | 6.12 (2.89-12.9) | 32/82 | 1.60 (1.08-2.38) | 45/69 | 2.07 (1.47-2.91) |
| | High | 15/72 | 9.37 (4.55-19.3) | 21/66 | 1.47 (0.92-2.34) | 36/51 | 2.28 (1.58-3.31) |

¹Multivariate model controlled for age at diagnosis, diabetes after blood draw, smoking status, and year from baseline to PC diagnosis;

TABLE 4
Adjusted hazard ratio (HR)¹ and 95% confidence interval (95% CI) of prostate cancer-specific
mortality and other cause of death in the two cohorts of men with localized disease

| | Discovery Set | | Validation Set | | | |
|---|---|---|---|---|---|---|
| | PHS (1982-2010) | | HPFS (1993-2010) | | Combined Sets | |
| D'Amico | Fatal/ | HR | Fatal/ | HR | Fatal/ | HR |
| risk | censored | (95% CI) | censored | (95% CI) | censored | (95% CI) |

PC Death

| | | | | | | | |
|---|---|---|---|---|---|---|---|
| BMI | | | | | | | |
| <25 kg/m² | Low | 9/184 | 1.00 (ref) | 1/212 | 1.00 (ref) | 10/396 | 1.00 (ref) |
| | Intermediate | 15/118 | 2.35 (1.03-5.37) | 8/166 | 9.19 (1.15-74) | 23/284 | 3.01 (1.43-6.34) |
| | High | 13/77 | 3.05 (1.30-7.17) | 14/89 | 24.4 (3.25-190) | 27/166 | 5.47 (2.64-11.3) |
| ≧25 kg/m² | Low | 5/107 | 1.15 (0.39-3.45) | 12/275 | 9.38 (1.22-72.39) | 17/382 | 2.25 (1.07-5.15) |
| | Intermediate | 13/81 | 2.91 (1.24-6.83) | 8/186 | 8.77 (1.09-70.4) | 21/267 | 3.32 (1.56-7.08) |
| | High | 23/46 | 7.82 (3.59-17.0) | 13/127 | 21.3 (2.77-163) | 36/173 | 8.61 (4.25-17.4) |
| C-peptide | | | | | | | |
| Q1-3 | Low | 8/221 | 1.00 (ref) | 7/362 | 1.00 (ref) | 145583 | 1.00 (ref) |
| | Intermediate | 18/160 | 2.74 (1.19-6.31) | 10/275 | 1.78 (0.68-5.68) | 28/435 | 2.25 (1.20-4.22) |
| | High | 26/85 | 6.59 (2.79-14.6) | 22/158 | 6.24 (2.65-14.7) | 48/243 | 6.35 (3.55-11.4) |
| Q4 | Low | 6/70 | 2.97 (1.02-8.64) | 6/125 | 3.16 (1.06-9.44) | 12/195 | 2.99 (1.39-6.40) |
| | Intermediate | 10/39 | 6.51 (2.55-16.6) | 6/77 | 4.51 (1.49-14.8) | 16/116 | 5.55 (2.73-11.3) |
| | High | 10/38 | 8.05 (3.12-20.8) | 5/58 | 4.67 (1.48-14.8) | 15/96 | 6.21 (3.02-12.8) |
| Energetic risk | | | | | | | |
| Low | Low | 10/248 | 1.00 (ref) | 5/381 | 1.00 (ref) | 15/629 | 1.00 (ref) |
| | Intermediate | 21/170 | 2.68 (1.26-5.70) | 10/280 | 2.52 (0.86-7.39) | 31/450 | 2.60 (1.40-4.82) |
| | High | 27/105 | 5.47 (2.64-11.3) | 21/162 | 8.20 (3.07-21.9 | 48/267 | 6.38 (3.56-11.4) |
| High | Low | 4/43 | 3.37 (1.05-10.8) | 8/106 | 6.97 (2.26-21.5) | 12/149 | 5.04 (2.35-11.4) |
| | Intermediate | 7/29 | 5.66 (2.14-15.0) | 6/72 | 6.96 (2.10-23.1) | 13/101 | 6.12 (2.89-12.9) |
| | High | 9/18 | 10.8 (4.25-27.3) | 6/54 | 9.5 (2.89-31.3) | 15/72 | 9.37 (4.55-19.3) |

Other Death

| | | | | | | | |
|---|---|---|---|---|---|---|---|
| BMI | | | | | | | |
| <25 kg/m² | Low | 49/144 | 1.00 (ref) | 35/178 | 1.00 (ref) | 84/322 | 1.00 (ref) |
| | Intermediate | 43/90 | 1.28 (0.85-1.93) | 31/143 | 0.87 (0.53-1.41) | 74/233 | 1.12 (0.82-1.53) |
| | High | 37/53 | 1.83 (1.19-2.81) | 23/80 | 1.01 (0.59-1.72) | 60/133 | 1.45 (1.04-2.03) |
| ≧25 kg/m² | Low | 39/73 | 1.77 (1.16-2.71) | 40/247 | 0.75 (0.47-1.19) | 79/320 | 1.17 (0.85-1.60) |
| | Intermediate | 30/64 | 1.15 (0.73-1.81) | 49/145 | 1.30 (0.83-2.02) | 79/209 | 1.32 (0.97-1.81) |
| | High | 25/44 | 1.65 (1.01-2.67) | 28/112 | 1.13 (0.68-1.87) | 53/156 | 1.43 (1.01-2.03) |
| C-peptide | | | | | | | |
| Q1-3 | Low | 55/174 | 1.00 (ref) | 50/319 | 1.00 (ref) | 105/493 | 1.00 (ref) |
| | Intermediate | 57/121 | 1.22 (0.84-1.77) | 57/228 | 1.34 (0.91-1.96) | 114/349 | 1.28 (0.98-1.67) |
| | High | 42/69 | 1.63 (1.09-2.45) | 34/146 | 1.29 (0.83-2.00) | 76/215 | 1.46 (1.08-1.96) |
| Q4 | Low | 33/43 | 2.34 (1.50-3.64) | 25/106 | 1.65 (1.01-2.69) | 58/149 | 1.99 (1.43-2.75) |
| | Intermediate | 16/33 | 1.42 (0.81-2.50) | 23/60 | 1.93 (1.16-3.12) | 39/93 | 1.72 (1.19-2.50) |
| | High | 20/28 | 2.53 (1.50-4.28) | 17/46 | 1.97 (1.13-3.43) | 37/74 | 2.21 (1.50-3.22) |
| Energetic risk | | | | | | | |
| Low | Low | 68/190 | 1.00 (ref) | 56/330 | 1.00 (ref) | 124/520 | 1.00 (ref) |
| | Intermediate | 63/128 | 1.15 (0.82-1.63) | 58/232 | 1.21 (0.84-1.76) | 121/360 | 1.19 (0.93-1.53) |
| | High | 54/78 | 1.79 (1.25-2.56) | 38/145 | 1.24 (0.82-1.89) | 92/223 | 1.51 (1.15-1.99) |
| High | Low | 20/27 | 2.84 (1.70-4.73) | 19/95 | 1.24 (0.73-2.10) | 39/122 | 1.78 (1.23-2.58) |
| | Intermediate | 10/26 | 1.16 (0.60-2.27) | 22/56 | 1.83 (1.11-3.04) | 32/82 | 1.60 (1.08-2.38) |
| | High | 8/19 | 1.21 (0.57-2.55) | 13/47 | 1.68 (0.91-3.09) | 21/66 | 1.47 (0.92-2.34) |

¹ Multivariate model controlled for age at diagnosis, baseline smoking status, diabetes after blood draw, year from baseline to PCa diagnosis, and C-peptide assay batch (only in models involved C-peptide). Follow-up between time of diagnosis to PCa death, censored or the end of study (Mar. 31, 2010)

TABLE 5
The C-index show significant improvement of predictability for death
from PC from model with the modified D'Amico risk alone to models
incorporating BMI, C-peptide, and the energetic risk among overall
PC patients and among men with low-intermediate risk cancer.

| | PC Death | | Other Death | | All Death | |
|---|---|---|---|---|---|---|
| | C- | P- | C- | P- | C- | P- |
| Prediction score | index | value | index | value | index | value |
| Overall | | | | | | |
| D'Amico risk | 0.72 | | 0.72 | | 0.70 | |
| D'Amico & BMI | 0.73 | 0.35 | 0.72 | 0.55 | 0.71 | 0.26 |
| D'Amico & C-peptide | 0.77 | 0.001 | 0.73 | 0.70 | 0.72 | 0.02 |
| D'Amico & energetic risk | 0.78 | 0.000 | 0.73 | 0.44 | 0.72 | 0.006 |
| Low-intermediate risk | | | | | | |
| D'Amico risk | 0.66 | | 0.72 | | 0.70 | |
| D'Amico & BMI | 0.67 | 0.45 | 0.72 | 0.60 | 0.71 | 0.24 |
| D'Amico & C-peptide | 0.71 | 0.19 | 0.73 | 0.15 | 0.71 | 0.10 |
| D'Amico & energetic risk | 0.74 | 0.048 | 0.72 | 0.20 | 0.71 | 0.07 |

In this prospective study of men with localized PC, high energetic risk (elevated BMI and/or plasma C-peptide level) was significantly associated with risk of PC-specific mortality independent of the modified D'Amico risk. Incorporating energetic risk into D'Amico risk significantly improved its predictability and reclassified 20% of the men into a higher risk of PC-specific mortality and all-cause mortality at a level that was similar to the original D'Amico high-risk group. The observed associations were strong and consistent between the two cohorts, minimizing the possibility of chance findings. The use of prospective measurements also supports a causal relationship. In both cohorts, elevated C-peptide levels were also significantly associated with 50-80% higher risk of deaths from other causes in both lean and overweight men independent of age, smoking status, diabetes, and the D'Amico risk.

### Example 3

### Exemplary Mortality Risk Score Determination

Among 843 men with baseline (1982) plasma levels of C-peptide who were subsequently diagnosed with PCa in the Physicians' Health Study, 614 (119 fatal PCa) men with baseline PSA were used as a training set and 689 (109 fatal PCa) men with PSA at diagnosis were used as a validation set. D'Amico risk score was defined by either baseline PSA (training set) or PSA at diagnosis (validation set) together with Gleason score and clinical stage at diagnosis (low=PSA≦10, Gleason≦6 and T1/T2, intermediate=PSA 10-20 or Gleason=7, and high=PSA>20, Gleason≧8, or T3). Each of the three D'Amico scores were subsequently divided by baseline C-peptide (low=quartiles 1-3 vs. high=quartile 4) to form a new mortality risk score (1-6) and the risk prediction was compared between the two scores using Cox regression model and KM-survival curve controlling for age at diagnosis.

A modified D'Amico risk score (1-3)—excluding clinical stage T4/N1/M1 was used: D'Amico score=3 if PSA>20 OR Gleason 8+OR T3;

D'Amico score=2 if PSA 10-20 OR Gleason 7;

D'Amico score=1 if PSA<10 AND Gleason 6 or less AND (Clinical T1 or T2). Because plasma C-peptide independently predict fatal prostate cancer, C-peptide levels (low=quartiles 1-3 vs. high=quartile 4) were incorporated into the three D'Amico risk score to create n exemplary mortality risk score (1-6):

1 if D'Amico score=low & C-peptide=low

2 if D'Amico score=low & C-peptide=high

3 if D'Amico score=intermediate & C-peptide=low

4 if D'Amico score=intermediate & C-peptide=high

5 if D'Amico score=high & C-peptide=low

6 if D'Amico score=high & C-peptide=high

Mortality scores of 4 & 6 significantly improved the risk prediction of D'Amico score of intermediate or high risk group. In both the training and validation sets, elevated baseline C-peptide levels (highest vs. lowest quartile) significantly predisposed men to higher risk of fatal PCa independent of age, PSA, and Gleason score or D'Amico risk score. The strongest association was seen in men with baseline PSA<4 ng/ml suggesting that high C-peptide levels predispose men to fatal outcome even before they had clinically detectable tumor. Among men with local or regional disease (clinical stage T1-3), D'Amico risk score significantly predicted risk of fatal PCa; the HRs (95% CI) for low, intermediate, and high score were 1.0 (ref), 3.8 (2.1-7.2), and 7.5 (4.1-13.9). The risk prediction was significantly improved when using the new mortality risk score; the HRs (95% CI) for mortality risk scores 1-6 were 1.0 (ref), 1.6 (0.6-4.4), 2.7 (1.1-6.3), 9.8 (4.3-22), 7.7 (3.6-16.5), and 12.6 (5.0-31). All these estimates were replicated using the validation set with very similar trends and magnitude of effects.

Thus, prediagnostic levels of C-peptide significantly and independently predicted future risk of fatal PCa, especially in men with baseline PSA<4 ng/ml, supporting a biological role of insulin/C-peptide in promoting aggressive neoplastic behavior even before clinical manifestation. The mortality risk score combining C-peptide with D'Amico risk score dramatically improved the risk prediction in men with local or regional PCa. It also can identify a subgroup of men with high C-peptide in the intermediate-high D'Amico risk score groups who could benefit the most from insulin/C-peptide-lowering treatment, thus the mortality risk score can be used as a screening tool in clinical trials.

### Example 4

### Assessment of Fasting and 2-h Post-Prandial Plasma and Urinary C-Peptide

Blood and urine samples were collected among 7-8 volunteers before (fasting) and two hours after a standard breakfast (bagel with egg and tomato, 1 bottle orange juice). In these subjects, fasting blood C-peptide was more highly correlated with ambient glycemia (r²=−0.86) than post-prandial C-peptide (r²=−0.11). However, post-prandial plasma C-peptide was a better marker for insulin (r²=0.96) or proinsulin (r²=1.0) production than fasting C-peptide. These data demonstrate that 1) urinary C-peptide is a reasonable marker for insulin and proinsulin in both fasting state (r range: 0.68-0.86) and the two hour post-prandial state (r²=0.61-0.64); and 2) urinary C-peptide correlates well with blood C-peptide in the two hour post-prandial state (r²=0.61), but is more variable in the fasting state (r²=0.14). The sensitivity of the ELISA assay for urinary C-peptide is 0.012 ng/ml, and CV %<4.1%.

### Example 5

### Reproducibility and Influence of Sample Processing Time

Quality Control of urinary C-peptide assay was assessed with the following results:

1. Blinded split: The CVs of 4.1% for plasma C-peptide and 5.9% for urinary C-peptide and the correlation between plasma and urinary C-peptide, r²=0.45 (n=7 IDs);

2. Processing methods for urine C-peptide: This pilot included 12 donors (3 samples per donor) and two quality control splits. For the donors, one sample was processed immediately after collection, the second sample was shipped to the laboratory with an ice pack and processed 24 hours after collection, and the third sample was shipped to the laboratory with an ice pack and processed 48 hours after collection. Results: mean CV for quality control samples: 5%; mean CV for donor samples (across 3 processing times): 6%; the spearman correlations: 0-24 hours, r²=0.93; 0-48 hours: r²=0.99; ICC=0.99.

3. Correlations between plasma and urinary C-peptide among 12 donors who provided both samples was r²=0.57 (n=12). The correlation was much stronger for non-fasting samples: r²=0.68 (n=9). This is consistent with above mentioned findings in Example 3 that urinary C-peptide correlates well with blood C-peptide in post-prandial state (r²=0.61) but not in fasting state (r²=0.14).

### Example 6

### Long-Term within-Person Reproducibility

In healthy men in the Health Professionals Follow-up Study, plasma C-peptide levels were strongly correlated with insulin levels in both fasting (correlation coefficient r²=0.83; n=249) and non-fasting (r²=0.87; n=183) status. In the same study, the within-person correlation coefficient for plasma C-peptide levels measured 4 years apart was reasonably good (r²=0.57). The long-term within-person reproducibility of urinary C-peptide was 0.67 for urinary C-peptide assessed among 40 women who provided two first morning urine samples, one year apart. These data demonstrate that levels of plasma or urinary C-peptide are stable over long durations of freezing: no degradation in any of the assays in sequential analysis of the blinded quality control samples was detected.

### Example 7

### Post-Prandial C-Peptide as a Sensitive Clinical Indicator of Treatment Efficacy by Metformin in Men Who Underwent Androgen Deprivation Therapy (ADT)

A phase 2/3 biomarker and safety study of palliative treatment of advanced prostate cancer was performed. Subjects were randomized between standard castration or castration plus metformin, with 30 patients/arm. Subjects were treated with medical or surgical castration for all subjects based on treating physician preference, with the randomized addition of oral metformin or not.

Outcomes monitored included fasting and post-prandial blood insulin and C-peptide levels.

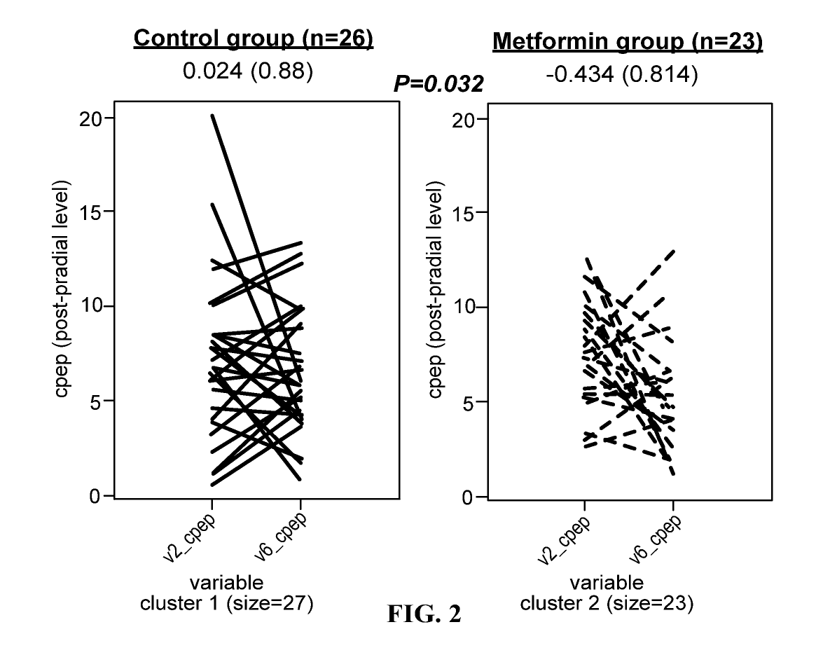

The results, shown in FIG. 2, demonstrated that post-prandial C-peptide is a sensitive biomarker (compared to insulin) in response to 6 month metformin treatment among men with androgen deprivation therapy; and that C-peptide can be a molecular target for identifying new drugs as therapeutic or chemopreventive agents that can lower circulating or tissue-specific C-peptide levels.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of evaluating risk of mortality for a subject with prostate cancer, the method comprising:
(i) providing a sample from the subject;
(ii) determining one or both of a level of C-peptide in the sample to obtain a test C-peptide value or determining the subject's body-mass-index ratio (BMI); and
(iii) determining the subject's D'Amico risk by determining the subject's level of prostate-specific antigen (PSA), Gleason score, and clinical stage, wherein:
(a) a subject with a level of PSA less than 10 μg/mL, a Gleason score equal to or less than 6, and a clinical T1 or T2 stage has a low D'Amico risk;
(b) a subject with a level of 10-20 μg PSA/mL, a Gleason score of 7, or a clinical T1 or T2 stage has an intermediate D'Amico risk; and
(c) a subject with a level of greater than 20 μg PSA/mL, a Gleason score equal to or greater than 8, or a clinical T3 stage has a high D'Amico risk;
wherein a subject with a test C-peptide value above a reference level of C-peptide, a BMI equal to or greater than 25 kg/m², and an intermediate or high D'Amico risk has an elevated risk of mortality.

2. The method of claim 1, wherein a subject with a test C-peptide value is in the highest quartile compared to a level of C-peptide in a reference group, a BMI of equal or greater than 25 kg/m², and an intermediate or high D'Amico risk has an elevated risk of mortality.

3. The method of claim 1, wherein a subject with a BMI of equal or greater than 30 kg/m² and an intermediate or high D'Amico risk has an elevated risk of mortality.

4. The method of claim 1, wherein the risk of mortality is from prostate cancer.

5. The method of claim 1, wherein the risk of mortality is within a specified time period.

6. The method of claim 5, wherein the specified time period is ten years.

7. The method of claim 1, wherein the sample comprises serum, plasma, whole blood, or urine.

8. The method of claim 1, wherein the subject is a human.

9. A method of predicting risk of developing fatal prostate cancer in a subject, the method comprising:
(i) providing a sample from the subject;
(ii) determining one or both of a level of C-peptide in the sample to obtain a test C-peptide value or determining the subject's body-mass-index ratio (BMI); and
(iii) determining the subject's D'Amico risk by determining the subject's level of prostate-specific antigen (PSA), Gleason score, and clinical stage, wherein:
(a) a subject with a level of PSA less than 10 μg/mL, a Gleason score equal to or less than 6, and a clinical T1 or T2 stage has a low D'Amico risk;
(b) a subject with a level of 10-20 μg PSA/mL, a Gleason score of 7, or a clinical T1 or T2 stage has an intermediate D'Amico risk; and
(c) a subject with a level of greater than 20 μg PSA/mL, a Gleason score equal to or greater than 8, or a clinical T3 stage has a high D'Amico risk;
wherein a subject with a test C-peptide value above a reference level of C-peptide, a BMI equal to or greater than 25 kg/m², and an intermediate or high D'Amico risk has an elevated risk of developing fatal prostate cancer.

10. The method of claim 9, wherein a subject with a test C-peptide value is in the highest quartile compared to a level of C-peptide in a reference group, a BMI of equal or greater than 25 kg/m², and an intermediate or high D'Amico risk has an elevated risk of developing fatal prostate cancer.

11. The method of claim 9, wherein a subject with a BMI of equal or greater than 30 kg/m² and an intermediate or high D'Amico risk has an elevated risk of developing fatal prostate cancer.

12. The method of claim 9, wherein the sample comprises serum, plasma, whole blood, or urine.

13. The method of claim 9, wherein the subject is a human.

14. A method of selecting an appropriate therapy for a subject with prostate cancer, the method comprising:
(i) providing a sample from the subject;
(ii) determining one or both of a level of C-peptide in the sample to obtain a test C-peptide value or determining the subject's body-mass-index ratio (BMI); and
(iii) determining the subject's D'Amico risk by determining the subject's level of prostate-specific antigen (PSA), Gleason score, and clinical stage, wherein:
(a) a subject with a level of PSA less than 10 μg/mL, a Gleason score equal to or less than 6, and a clinical T1 or T2 stage has a low D'Amico risk;
(b) a subject with a level of 10-20 μg PSA/mL, a Gleason score of 7, or a clinical T1 or T2 stage has an intermediate D'Amico risk; and
(c) a subject with a level of greater than 20 μg PSA/mL, a Gleason score equal to or greater than 8, or a clinical T3 stage has a high D'Amico risk;
wherein a subject with a test C-peptide value above a reference level of C peptide (e.g., in the highest quartile compared to a level of C-peptide in a reference group), a BMI equal to or greater than 25 kg/m² (e.g., above 30 kg/mg²), and an intermediate or high D'Amico risk has an elevated risk of developing fatal prostate cancer, and is treated aggressively.

15. The method of claim 14, wherein the aggressive treatment comprises one or more of surgery, radiation therapy, hormone therapy, chemotherapy, and biologic therapy.

16. A method of selecting an appropriate therapy for a subject with prostate cancer, the method comprising:
providing a sample from the subject;
determining a level of C-peptide in the sample; and
selecting a therapy comprising an anti-diabetic or insulin-lowering drug for a subject who has a level of C-peptide above, or at or above, a reference level, or selecting a therapy lacking an anti-diabetic or insulin-lowering drug for a subject who has a level of C-peptide below a reference level.

17. The method of claim 14, the method further comprising administering the selected therapy to the subject.

18. The method of claim 14, wherein the sample comprises serum, plasma, whole blood, or urine.

19. The method of claim 14, wherein the subject is a human.

20. The method of claim 14, the method further comprising administering the selected therapy to the subject.

21. The method of claim 16, wherein the anti-diabetic or insulin-lowering drug is selected from the group consisting of metformin, phenformin, buformin, and proguanil.

22. The method of claim 16, wherein the sample comprises serum, plasma, whole blood, or urine.

23. The method of claim 16, wherein the subject is a human.
